(19) United States
(12) Patent Application Publication
(10) Pub. No.: US 2025/0316347 A1
(43) Pub. Date: Oct. 9, 2025

(54) Lab Work App

(71) Applicant: Ian R Guyton, Lancaster, CA (US)

(72) Inventor: Ian R Guyton, Lancaster, CA (US)

(21) Appl. No.: 18/629,659

(22) Filed: Apr. 8, 2024

(51) Int. Cl. G16H 10/40

(52) U.S. Cl. CPC G16H 10/40

(57) ABSTRACT

I get lab work done regularly, but one day I thought something to the effect of I can see a user friendly lab work app that could be used from the comfort and convenience of a personal computer at home. Working with this lab work app, I can see a device that attaches to a personal computer like a printer that is used for blood and urine samples and is conveniently sized for a pc. The blood extracting process may be done with the help of a nurse or medical professional from a house call.

### CROSS-REFERENCE TO RELATED APPLICATIONS

Primex Clinical Laboratories, Labcorp.

### STATEMENT REGARDING FEDERALLY SPONSORED RESEARCH OR DEVELOPMENT

N/A yet.

### THE NAMES OF THE PARTIES TO A JOINT RESEARCH AGREEMENT

N/A yet.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

N/A yet.

### STATEMENT REGARDING PRIOR DISCLOSURES BY THE INVENTOR OR A JOINT INVENTOR

Disclosed idea to thoughtstopaper.com on around Oct. 13, 2021, patent pending expired on May 31, 2023.

### BACKGROUND OF THE INVENTION

Medical diagnostic tests refer to a variety of physical examinations issued by medical professionals to confirm the presence of illness or underlying disease in patients. A common form of medical diagnostic testing is done through collecting samples of blood or urine from the patient, which are then reviewed by a medical professional for further analysis and diagnosis. This form of testing typically requires patients to visit a healthcare professional or medical lab to provide or drop off a sample. However, this creates a common issue for many patients that have trouble traveling to and from the house (such as a medical ailment that affects mobility, or simply even non-medical related transportation issues). The present invention offers a solution for patients to provide blood or urine samples to their medical providers from the comfort and convenience of their own home.

### FIELD OF THE INVENTION

The present invention relates generally to medical diagnostics. More specifically, a method and apparatus that is used to analyze blood and urine samples.

### DESCRIPTION OF RELATED ART INCLUDING INFORMATION DISCLOSED UNDER 37 CFR 1.97 AND 1.98

1 Primex Clinical Laboratories provides clinical diagnostics testing services to thousands of clients throughout California, Nevada and American Samoa. 2. Labcorp is a leading global life sciences company. With a mission to improve health and improve lives, Labcorp delivers world-class diagnostic solutions, brings innovative medicines to patients faster and uses technology to improve the delivery of care.

### BRIEF SUMMARY OF THE INVENTION

The “Lab Work App” and device solves a problem of mobility and convenience. Two advantages are users can go to their Doctor's appointment with a more informed opinion of their health needs (preventive and diagnostic), and this could be done from the comfort and convenience of the user's home.

### DETAILED DESCRIPTION OF THE INVENTION

The present invention, herein referred to as “Lab Work App”, comprises a software application and device that attaches to a personal computer, similar to how a printer does. Instead of a device which prints paper however, this device reviews blood and urine samples with technology that is used to analyze blood and urine lab work from the comfort and convenience of the user's own home.

The blood extracting process may be done with the help of a nurse or medical professional from a house call. The user-friendly software application processes the blood and urine samples and checks HDL, LDL, iron, glucose levels, etc., then organizes results that can then be emailed to the user's doctor for further review and treatment.

Although the invention has been explained in relation to its preferred embodiment, it is to be understood that many other possible modifications and variations can be made without departing from the spirit and scope of the invention.

### BRIEF DESCRIPTION OF THE SEVERAL VIEWS OF THE DRAWING(S)

FIG. 1a shows a “Lab Work App” and device user was feeling ill and made a house call to a medical professional to do lab work.

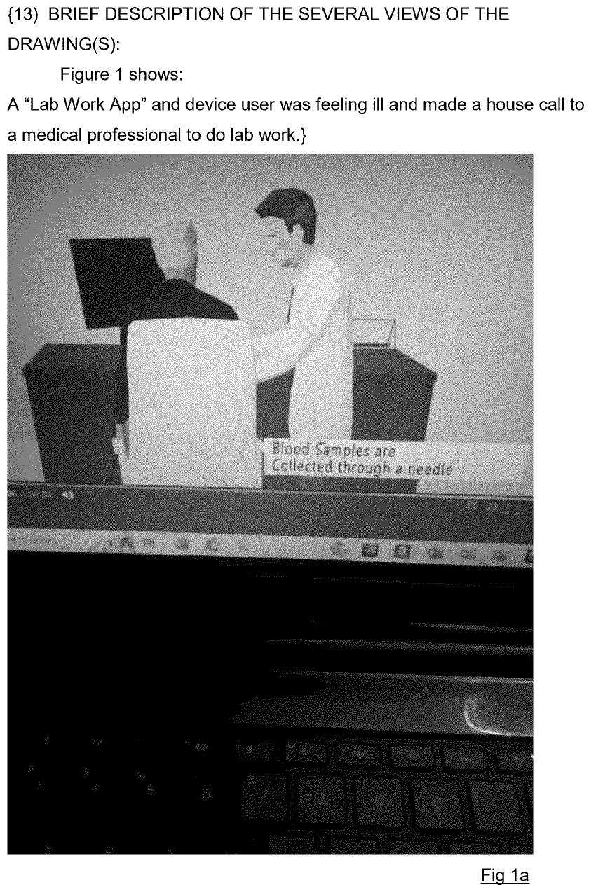

FIG. 1b shows the medical professional then uploads the blood sample into a device connected to a computer.

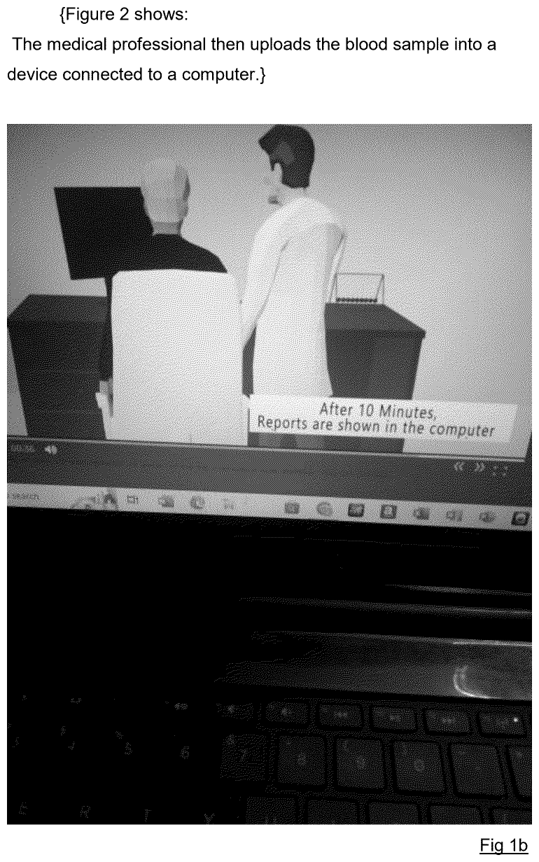

FIG. 1c shows the results are then displayed on the app.

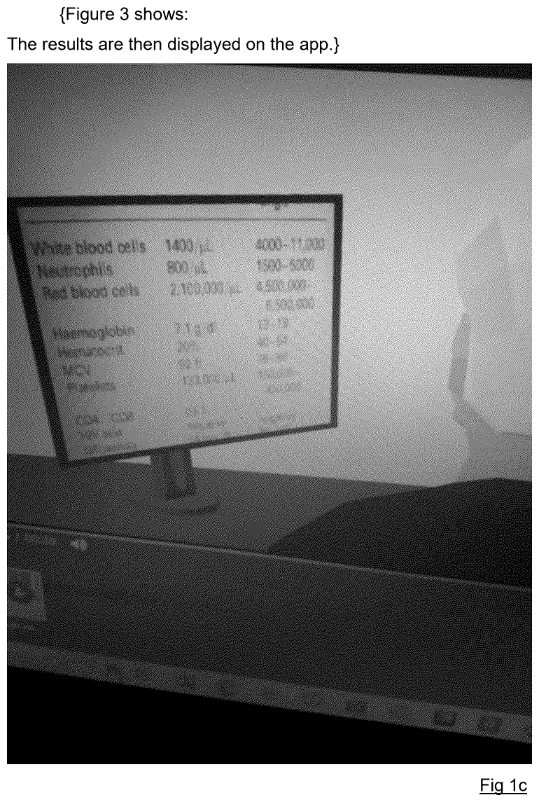

For this “Lab Work App” and device innovation the financial profiteers could be:
The product manufacturers get paid on average $30,000 that includes the cost of designing, prototyping, testing, and launching the new product.
The medical insurers may get paid because they have more business with this preventive and diagnostic “Lab Work App” and device that comes from their clients having awareness of health problems that they did not have before this product.
Stores that sell this “Lab Work App” and device product may split the profit between the inventor 60/40 percent with the latter getting the lesser but hope to move to 50/50.
Stores that sell nutritious groceries and vitamin supplements may see an increase in sales as more “Lab Work App” and device clients become aware of their health needs.
Lab testing can be expensive, with or without insurance, the cost can range anywhere between $100 to over $1000 making this “Lab Work App” and device a frugal option for patients because this product may potentially pay for itself.

1. App and device designed for personal computers.

2. Blood extraction done by medical professional in response to house call.

3. For medical preventive and diagnostic testing.
